(19) United States
(12) Patent Application Publication
(10) Pub. No.: US 2026/0279508 A1
(43) Pub. Date: Sep. 17, 2026

(54) NEW INTEGRATED ANALYTICAL PLATFORM FOR THE ASSESSMENT OF THE QUALITY OF COMPLEX NATURAL PRODUCTS

(71) Applicant: ABOCA S.P.A SOCIETÀ AGRICOLA, Sansepolcro (AR) (IT)

(72) Inventors: Valentina MERCATI, Sansepolcro (AR) (IT); Emiliano GIOVAGNONI, Sansepolcro (AR) (IT); Mattia GIANNI, Urbania (PU) (IT); Michela BURICO, Sansepolcro (AR) (IT); Luisa MATTOLI, Sansepolcro (AR) (IT); Valentino MERCATI, Sansepolcro (AR) (IT)

(73) Assignee: ABOCA S.P.A SOCIETÀ AGRICOLA, Sansepolcro (AR) (IT)

(21) Appl. No.: 19/469,799

(22) PCT Filed: Mar. 27, 2024
(86) PCT No.: PCT/IB2024/052960

§ 371 Date: Sep. 26, 2025

(30) Foreign Application Priority Data
Mar. 28, 2023 (IT) 102023000005949

(51) Int. Cl. G16C 20/30; G16C 60/00

(52) U.S. Cl. CPC G16C 20/30; G16C 60/00

(57) ABSTRACT

The present invention provides a new integrated platform that allows the validation of production processes, the standardization and the evaluation of complex natural products, allowing the characterization of the complex mixture contained within products with a therapeutic and/or healthy effect based on or consisting of from natural substances “as an entire system”, without limiting their characterization to a reductionistic chemical approach aimed at a single marker, but rather integrating qualitative-quantitative data and fingerprint characterization metabolomics of the mixture with physical and biophysical activity data thus providing a characterization of the product which is the basis of the validation process of the production process which allows to guarantee reproducibility between different batches of this product in terms of efficacy and safety. The analytical platform described here allows us to guarantee the complex natural product as a whole, taking into consideration all its quality and efficacy properties in order to guarantee the reproducibility of the effect and safety through a multidimensional analytical approach.

### TECHNICAL FIELD OF THE INVENTION

The present invention concerns the validation of production processes, standardization and evaluation of complex natural products. In particular, the invention provides a new integrated platform that allows to validate, standardize and evaluate complex natural products, allowing to characterize the complex mixture used for the production of products with therapeutic effects (e.g. medical devices) or that assist the physiological functions of the organism (eg food supplements) based on or constituted by natural substances “as an entire system”, without limiting its characterization to a chemical approach aimed at a single marker. The platform allows to integrate qualitative and quantitative fingerprint characterization data metabolomics of the mixture with physical and biophysical activity data, thus providing a characterization of the product which is the basis of the validation process of the production process which allows to guarantee reproducibility in terms of efficacy and safety between different batches of such product. The analytical platform described herein allows to guarantee the quality of the complex natural product as a whole, taking into consideration the totality of its properties in order to guarantee the reproducibility of the effect and safety through a multidimensional analytical approach.

### STATE OF THE ART

In recent years, the family of complex natural products has become increasingly important in the therapy of so-called “self-limiting diseases” (e.g. cough, sore throat, constipation, etc.), for particular groups of patients (pediatric and geriatric age for example), as well as for important “unmet medical needs”, in particular syndromes and functional disorders, for which the classic pharmacological approach is unable to provide satisfactory answers in terms of benefit-risk ratio. This last parameter, which plays a crucial role in defining the therapy, has evolved in recent years and has gone from an evaluation aimed exclusively at the patient to one that also evaluates the effect of the therapeutic treatment on the environment. This is because the effects of introducing non-biodegradable substances into the environment causes direct and measurable negative effects on human health, creating an unsustainable vicious cycle.

It is clear that in this new context, which could be defined as a “benefit-risk relationship extended to man and the environment”, the use of natural substances, biodegradable by definition, represents an opportunity to be explored in all its potential usefulness.

Regulation 2017/745 relating to medical devices has provided for the possibility of using, among others, complex natural products for the treatment and prevention of pathologies and has created a defined regulatory framework that is added to the already known one for drugs. The substantial difference between the two regulatory contexts is linked to the mechanism of action through which the therapeutic activity is achieved: in the case of the drug this must be obtained through a pharmacological (or immunological, or metabolic) action which translates in an interaction of a specific substance and a cellular target (usually defined as a receptor), while in the case of medical devices the therapeutic objective must be obtained with any mechanism of action other than those reported above. Therefore, pursuant to this legislation, therapeutic compositions/formulations are defined as drugs or medical devices based on the mechanism of action with which they exert their therapeutic effect. Coming to the case of natural substances with therapeutic effects, these can be classified both as drugs and as medical devices. Nonetheless, in the case of the drug it is necessary to demonstrate that the therapeutic activity is achieved through a specific substance contained in the natural substance (generally defined as a marker or active ingredient) which acts on a specific cellular target, while in the case of medical devices this must be connected to the totality of the complex matrix and cannot be traced back to a specific mechanism of action.

From a technical and regulatory point of view this translates into the fact that drugs, considering that the therapeutic action is carried out by a single molecule or a combination of molecules through a pharmacological, immunological or metabolic mechanism of action (Article 2 of Directive 2001/83) they must be able to restore, correct or modify physiological functions, while medical devices, which must act with a non-pharmacological, immunological or metabolic action, must therefore modify a physiological or pathological process or state (article 2 Regulation 2017/745) [Capone, L., Geraci, A., Giovagnoni, E., Marcoaldi, R., and Palazzino, G. (2012) “Elementi di valutazione e di discernimento tra dispositivo medico e medicinale. Roma: Istituto Superiore di Sanità. Rapporti ISTISAN 12/30; Racchi, M., Govoni, S., Lucchelli, A., Capone, L., and Giovagnoni, E. (2016). Insights into the definition of terms in European medical device regulation. Expert Rev. Med. Devices 13 (10), 907-917. doi: 10.1080/17434440.2016. 1224644].

In the context of the medical device composed of natural substances, where the therapeutic action of the natural product is due to the set of components that characterize it, it appears necessary to develop criteria for the characterization and validation of these innovative products in order to guarantee the reproducibility of the benefit ratio-risk underlying their therapeutic use.

Given the variability of natural substances, it is necessary to be able to provide production processes and quality control approaches that are able to guarantee that the single batch of product falls within a defined range of variability, determined experimentally, and validated as capable of guaranteeing the maintenance of the demonstrated risk-benefit ratio.

This characterization must be placed at the basis of the demonstration of the effectiveness and safety of the medical device, in accordance with the provisions of Regulation 2017/745, requirement 13.3, Annex I of and must be reported in the product labeling as a guarantee of its characterization.

This result can be achieved through an approach that is not limited to determining the composition of the product from a quantitative chemical point of view. In fact, if this approach is sufficient to characterize a product made up of a purified substance, it is not able to define the reproducibility of a complex substance for which it is necessary to be able to describe the product from a physico-chemical and biological point of view.

In accordance with what was said above, also according to the complex substances obtained from natural biological matrices (UVCB Substances of Unknown or Variable composition, Biological materials) are not definable like pure chemical substances, but rather through analytical techniques, such as fingerprinting and physical and biological characterization, which are able to define and validate the variability of the complex substance within specific ranges.

Therefore, integrated and sophisticated analytical approaches are needed to characterize their structure and effectiveness performance, as well as to define the correlation between the various parameters.

Correlation studies between fingerprint data and the activities of complex natural products are starting to appear in the literature. For example, in publication Jing Wang, Hongwei Kong, Zimin Yuan, Peng Gao, Weidong Dai, Chunxiu Hu, Xin Lu, Guowang Xu, A novel strategy to evaluate the quality of traditional Chinese medicine based on the correlation analysis of chemical fingerprint and biological effect. Journal of Pharmaceutical and Biomedical Analysis 83 (2013) 57-64, an analysis of the correlation between the fingerprint of a certain number of compounds included in complex natural products and their biological effect is described in order to identify a certain number of marker components of quality of such products in relation to their biological effect. However, even in these studies, the standardization is on single markers.

There are currently no procedures available that allow the complex mixture contained within natural products to be characterized as an entire “system”, without therefore limiting the characterization to a few selected markers, which can guarantee the validation of the production process and/or the verification of reproducibility between different batches of complex natural products. Furthermore, the absence of these procedures does not allow us to ensure the reproducibility of the risk-benefit ratio in clinical studies.

### SUMMARY OF THE INVENTION

The present invention provides an innovative analytical platform which, through the integration of advanced analytical techniques, allows characterizing the complex mixture contained within natural products “as an entire system”, without limiting its characterization to a reductionistic chemical approach. aimed at a single marker. This is done by integrating qualitative-quantitative and fingerprint characterization data metabolomics of the mixture with physical and biophysical activity data. The authors of the invention have managed to provide a characterization of products, such as a product which is the basis of the validation procedure of the production process, which allows to guarantee reproducibility between different batches of this product.

The analytical platform described here allows us to guarantee the complex natural product as a whole, taking into consideration all its quality and efficacy properties in order to guarantee the reproducibility of the effect and safety through a multidimensional analytical approach.

In fact, a complex natural system expresses characteristics that cannot be described by a single technique. The present invention provides a method that allows the correlation of qualitative-quantitative and efficacy parameters, thus providing an evaluation of a complex product.

The present invention provides a process for characterizing a product having therapeutic and/or healthy properties, said product comprising complex natural systems (such as for example a composition obtained by mixing natural substances, their extracts and fractions, preferably obtained with low environmental impact processes through physical production processes, said composition being able to be in different pharmaceutical forms such as syrup, tablets, granule sachets, caps, spray, suspension, aerosol, aerosol powders), including the following steps:
a. carry out a qualitative-quantitative fingerprint analysis of a standard formulation of said product and of formulations that differ from said standard formulation due to the presence of the same ingredients of the same quality at different percentages, or due to the presence of the same ingredients of different quality at the same percentages, thus obtaining an X block of fingerprint data
b. carry out an analysis of one or more biophysical properties of a standard formulation of said product and of formulations that differ from said standard formulation due to the presence of the same ingredients of the same quality at different percentages, or by the presence of the same ingredients of different quality at same percentages, thus obtaining a data block Y of biophysical properties
c. carry out an integration by correlation of the data by means of multivariate analysis which involves the correlation of said data block X with said data block Y by integrating by correlation each of the data obtained in a. with each of the data obtained in b, thus obtaining a regression line for each pair of values for which the regression coefficient R² and the mean squared estimate error RMSEE are calculated, selecting those pairs of data wherein R²≥0.8 thus providing a model which allows predict each biophysical property of the standard formulation starting from the fingerprint used.

The present invention also provides a process for the production validation of one or more batches of a product having therapeutic and/or healthy properties comprising complex natural systems comprising the following steps:
a. carry out a qualitative-quantitative fingerprint analysis of a standard formulation of said product and of formulations that differ from said standard formulation due to the presence of the same ingredients of the same quality at different percentages, or due to the presence of the same ingredients of different quality at the same percentages, thus obtaining an X block of fingerprint data
b. carry out an analysis of one or more biophysical properties of a standard formulation of said product and of formulations that differ from said standard formulation due to the presence of the same ingredients of the same quality at different percentages, or by the presence of the same ingredients of different quality at same percentages, thus obtaining a Y block of biophysical property data
c. carry out an integration by correlation of the data using multivariate analysis which involves the correlation of said data block X with said data block Y, integrating by correlation of each of the data obtained in a. with each of the data obtained in b, thus obtaining a regression line for each pair of values for which the regression coefficient R² and the mean squared estimate error RMSEE are calculated, selecting those pairs of data wherein R²≥0.8 thus providing a model which allows to predict each biophysical property of the standard formulation starting from the fingerprint used
d. carry out a qualitative-quantitative fingerprint analysis with the same methods used in a. of one or more batches of said product to be validated, using the regression model developed in c. so as to predict the biophysical characteristics described in point b.

### Glossary

The term product having healthy and/or therapeutic properties comprising complex natural systems according to the present invention is a product made up of natural substances, such as a medical device according to EC Regulation 745/2017, requirement 13.3, Annex I (product having therapeutic properties), or a food supplement consisting of complex natural substances according to Directive 2002/46/EC, article 2 and DLgs 169/2004 article 2 (product with health-promoting activities).

In every part of the description and claims, said product is not intended as a product naturally existing in nature but as a product resulting from the formulation of complex natural substances. Therefore, the term “product having healthy and/or therapeutic activities including complex natural systems” can be replaced, in every part of the text and claims with “formulation having healthy and/or therapeutic properties comprising (or consisting of) complex natural systems” or with “composition having healthy and/or therapeutic properties comprising (or consisting of) natural systems complexes” or with “mixture having healthy and/or therapeutic properties comprising (or consisting of) complex natural systems”.

Furthermore, in any part of the description and claims, the expression “comprising complex natural systems” can be replaced by “consisting of complex natural systems”.

By product with healthy activities, it is meant a product, as defined above, whose intake by any suitable route benefits health or is beneficial to health, in particular it benefits the health of healthy people. They fall within the definition of products with healthy activities such as supplements, foods for special medical purposes, mixtures of nutrients such as proteins, vitamins, enzymes, oils, etc.

The term formulation or quality sample here also defined as “standard formulation” or “target product” of a product x, indicates a formulation of the product having therapeutic and/or healthy properties including complex natural systems as defined above, wherein said product responds to the characteristics necessary to be defined as a medical device according to EC Regulation 745/2017, requirement 13.3 Annex I, or as a food supplement made up of complex natural substances according to Directive 2002/46/EC article 2, and DLgs 169/2004 article 2. According the aforementioned regulation fall within the definition of medical devices are compositions with therapeutic effect consisting of substances or an association of substances, including materials of biological origin, which can therefore be in different pharmaceutical forms such as syrup, tablets, granulate sachets, caps, sprays, suspensions, aerosols, aerosol powders and the like. The same type of formulation can also have healthy compositions, such as food supplements as defined here.

The term poor quality formulation (or sample or ingredient) (of a product In the present invention, the formulations which differ from the standard formulation (or sample) due to the presence of the same ingredients at different percentages compared to said sample, or due to the use of the same ingredients present in the standard formulation, but having quality characteristics different from those used in the definition of product be defined as poor quality formulations.

Data integration indicates the construction of a correlation model between qualitative-quantitative fingerprint data and biophysical activity, thanks to which by measuring a piece of data (for example NIR fingerprint) on a new batch it is possible to predict the corresponding data (for example biophysical activity).

The acquisition of this information on many batches leads to defining very stringent quality ranges by taking into consideration multiple aspects according to a correlation model: the qualitative-quantitative fingerprint and the biophysical activity. Quality ranges can thus be specifically defined depending on the product of interest.

Characterization in this description: the term “characterization” means a qualitative-quantitative description (definition) of the complex natural product of interest and its biophysical properties.

Qualitative-quantitative description of the complex natural product: this means the result of carrying out product analysis using fingerprint techniques metabolomics such as NIR spectroscopy and/or mass spectrometry.

NIR spectroscopy: is the study of the absorption and emission of a ray of light in the infrared range (4000-12500 cm-1) by matter.

Mass spectrometry: is a method of studying and measuring a specific spectrum. In the case of mass spectrometry, the masses of a sample are measured through the mass-charge ratio, after passing through an electromagnetic field.

Description of the biophysical properties of the complex system: means the execution of tests that define biophysical properties of the product known to be correlated with the therapeutic efficacy of the product such as, for example, tests measuring the barrier effect, adhesion activity and antioxidant activity, neutralizing activity, hydrating activity, lubricating activity.

The term Poliresin in the present invention is used to indicate a plant complex based on polysaccharides, resins and flavonoids obtained by subjecting the three starting plants (Plantain, Grindelia, Helichrysum) to a physical extraction process with low environmental impact (LPME process, Liquid Phase Micro Extraction, a process characterized by the liquid phase extraction of plant parts reduced into microscopic particles) capable of producing extractive fractions and standardizing their content within the finished product.

OPLS (Orthogonal Partial Least Squares/Orthogonal projections to latent structures) in this description refers to the well-known method of multivariate data analysis that makes efficient use of all relevant data, with little loss of information. This is a recent extension, known in the literature, of PLS. OPLS separates the systematic variation of or parallel, separating it from the non-predictive or orthogonal one.

PLS (Projections to latent structures by means of partial least squares) is a well-known statistical method for relating two matrices X and Y through a multivariate linear model. PLS finds the linear or polynomial relationship between a block of data Y (dependent variables) and a block of variables X (predictor variables). The objective function of PLS is to maximize the covariance between X and Y.

PLS2 (PLS2=Partial Least Squares 2) was the first algorithm, now known in the literature, proposed to perform PLS regression, and is currently one of the most used implementations of PLS. PLS2 refers to a model with multiple dependent variables. Given the Y matrix of responses and the X matrix of predictors, the purpose of PLS2 is to calculate the regression coefficient matrix B that produces the linear regression model.

Complex natural ingredients according to this description are those ingredients present in products based on natural substances, therefore not synthetic, which include multiple constituents such as for example cut or pulverized plant parts, plant extracts or plant parts through hydroalcoholic extractions, fractions of said extracts, such as for example the fractions obtained by filtration on a semi-permeable membrane (microfiltration, ultrafiltration, nanofiltration) or those obtained by selective precipitation through the controlled evaporation of hydroalcoholic solutions, or those obtained by treatment on adsorption resins.

### DETAILED DESCRIPTION OF THE FIGURES

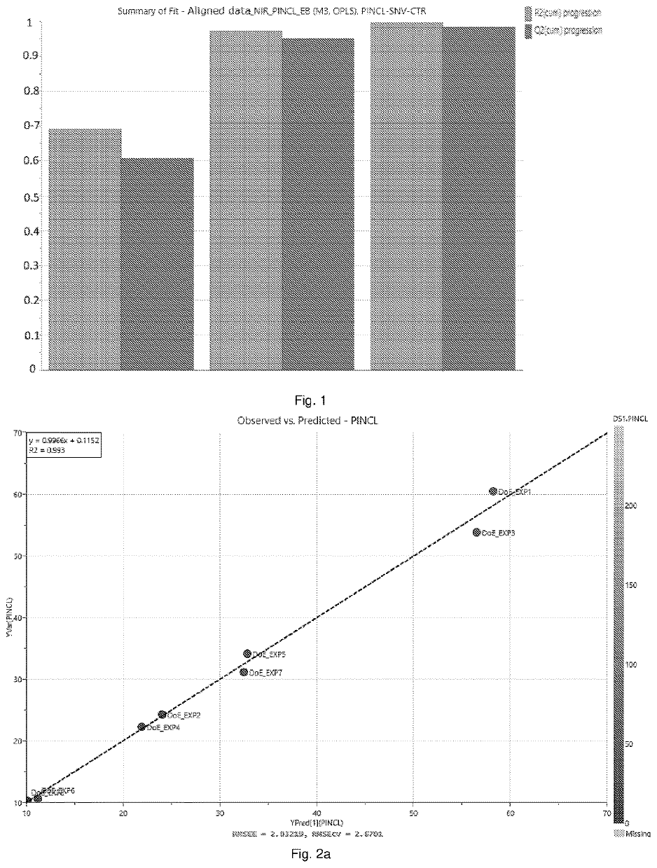

FIG. 1. R² and Q² of the OPLS model with NIR profile (fingerprint Near InfraRed, fingerprint or profile by near infrared spectroscopy) & Adhesive effect (PINCL).

The OPLS algorithm (Orthogonal Partial Least Squares/Orthogonal projections to latent structures, Orthogonal partial least squares/Orthogonal projections to latent structures) separates the systematic variation of the information better as it separates orthogonal information from parallel information and takes into consideration only the predictive or parallel information.

The information of block X is given by the NIR fingerprint, while the information of block Y is given by the adhesive effect (PINCL).

This figure shows the bar graph of the cumulative diagnostic parameters (summary of fit) R² and Q² (in light gray and dark grey, respectively) which express the performance of the OPLS model of correlation between the data NIR fingerprint and adhesive effect. R² is a measure that expresses the goodness of the model (goodness of fit) while Q² is a measure that expresses the goodness of the prediction (goodness of prediction). The test of the diagnostic parameters R² and Q² is considered satisfied the closer both parameters are to each other and tend towards 1 (y axis). The number of components used is reported on the abscissa (x-axis) and is indicated in square brackets. The two bars on the left represent the value of R² and Q² for the parallel component and 0 orthogonal components (comp [1+0]). The two bars in the center represent the value of R² and Q² for the parallel component and the 1st orthogonal component (comp [1+1]). The two bars on the right represent the value of R² and Q² for the parallel component and the 2nd orthogonal component (comp [1+2]).

In this case, therefore, the best OPLS model is the one that uses 1 parallel component and 2 orthogonal components (comp [1+2]) as both the values of R² and Q² are close to each other and tend to be 1.

The graph at the bottom right shows the software used and the reprocessing date.

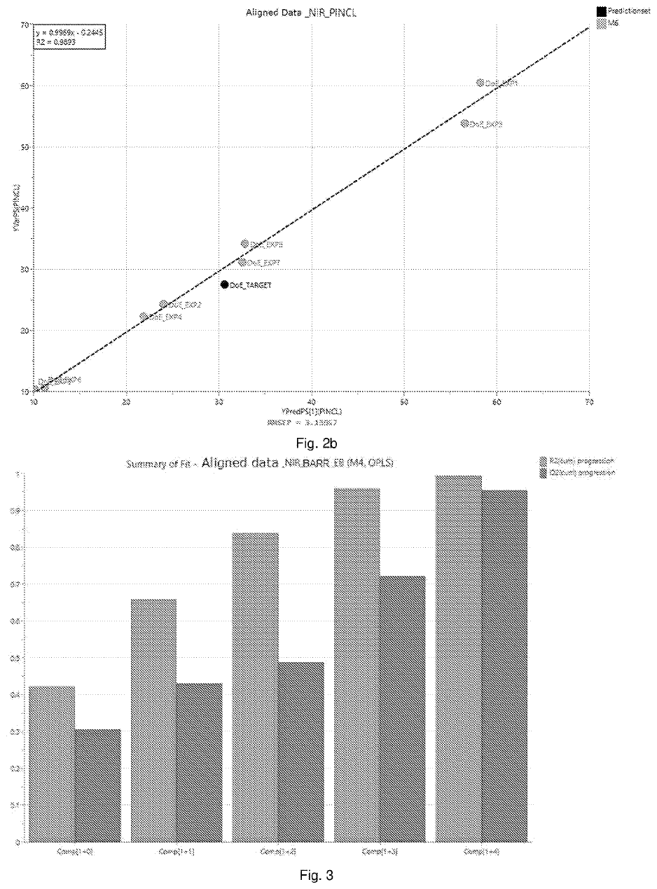

FIG. 2a. Scatter plot obtained using the OPLS algorithm (Orthogonal Partial Least Squares/Orthogonal projections to latent structures, Orthogonal partial least squares/Orthogonal projections to latent structures) of NIR fingerprint data (Near InfraRed) integrated with the adhesion values to the inclined plane (PINCL). The information of the block as predicted (or calculated) data on the abscissa (x-axis). Each circle shown in the graph represents the experimental measurement of the adhesion to the inclined plane of each sample analyzed (see table 7) and characterized by a different composition as described in the text (see table 6). The samples are identified by the acronym “DOE_EXP from 1 to 8” and correspond to the samples called “Experiment from 1 to 8” in table 7.

The dotted line in the center of the graph represents the relationship between the data and is characterized by the equation y=0.9966x+0.1152, with correlation coefficient R² equal to 0.993, shown in the graph at the top left.

This straight line expresses a low error value of the estimate, RMSEE equal to 2.03219 (Root Mean Square Error of Estimation, Root Mean Square Error of the estimate), and a low cross-validation error, RMSE_{CV} equal to 2.6701 (Root Mean Square Error of Cross Validation), which will result in a very accurate prediction of biophysical activity values (Y data block) starting from the NIR fingerprint (X data block).

The RMSEE and RMSE_{CV} values are reported under the heading YPRED[1](PINCL) on the x-axis. The vertical bar on the right expresses in color gradient the experimental values of the Y block used to create the model, in this case the measurement values of the adhesion to the inclined plane. The graph at the bottom right shows the software used and the reprocessing date.

FIG. 2b. Scatter plot obtained using the OPLS algorithm (Orthogonal Partial Least Squares/Orthogonal projections to latent structures, Orthogonal partial least squares/Orthogonal projections to latent structures) of NIR fingerprint data (Near InfraRed) integrated with the adhesion values to the inclined plane (PINCL) with prediction of the TARGET adhesion effect starting from the NIR fingerprint.

The graph is essentially that of FIG. 2a where in addition the TARGET sample (DoE_Target) is present in prediction.

Therefore all the details described in the previous graph remain valid. Furthermore, in this case the NIR fingerprint of the TARGET sample has been added to the information of block X, consisting of the NIR fingerprint.

The data of the samples used to build the model are represented by a light gray circle, while the TARGET sample in the prediction model is represented by a dark gray circle. By exploiting the regression model, the fingerprint information of the TARGET sample is used to predict the information of the Y block, in this case to predict the adhesion information to the inclined plane.

The dotted line in the center of the graph represents the interpolation between the data including the TARGET sample and is characterized by the equation y=0.9969x−0.2445, with correlation coefficient R² equal to 0.9893, shown in the graph at the top left. This line expresses a low prediction error value, with the RMSEP equal to 3.13957 (Root Mean Square Error of Prediction, root of the mean square error of prediction), which indicates a very accurate prediction of biophysical activity values.

The RMSEP value is reported under YPredPS [1](PINCL) on the x-axis.

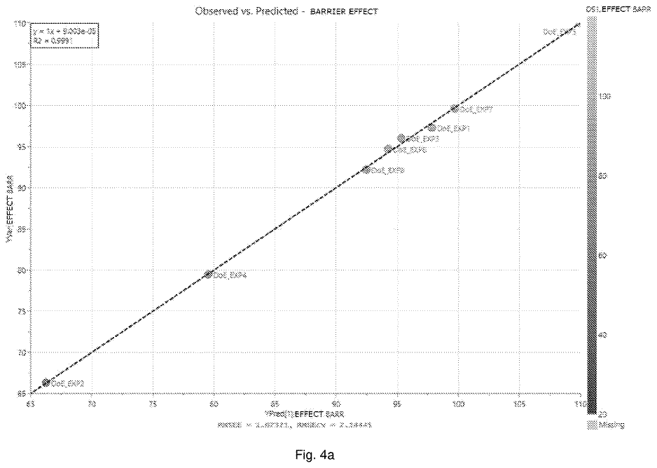

FIG. 3. R² and Q² of the OPLS model with NIR profile (fingerprint Near InfraRed, fingerprint or profile by near infrared spectroscopy) & Barrier effect.

The OPLS algorithm (Orthogonal Partial Least Squares/Orthogonal projections to latent structures, Orthogonal partial least squares/Orthogonal projections to latent structures) separates the systematic variation of the information better as it separates orthogonal information from parallel information and takes into consideration only the predictive or parallel information.

The information of block X is given by the NIR fingerprint, while the information of block Y is given by the barrier effect (BARR).

This figure shows the bar graph of the cumulative diagnostic parameters (summary of fit) R² and Q² (in light gray and dark grey, respectively) which express the model performance (model performance) of correlation between the data of NIR fingerprint and barrier effect. R² is a measure that expresses the goodness of the model (the goodness of fit) while Q² is a measure that expresses the goodness of the prediction (the goodness of prediction). The test of the diagnostic parameters R² and Q² is considered satisfied the closer both parameters are to each other and tend towards 1 (y axis). The number of components used is reported on the abscissa (x-axis) and is indicated in square brackets. The two bars on the left represent the value of R² and Q² for the parallel component and 0 orthogonal components (comp [1+0]). The bars in the center represent the value of R² and Q² for the parallel component and, respectively, the 1st, 2nd, 3rd orthogonal component (comp [1+1], comp [1+2], comp [1+3]). The last two bars on the right represent the value of R² and Q² for the parallel component and the 4th orthogonal component (comp [1+4]).

In this case, therefore, the best OPLS model is the one that uses 1 parallel component and 4 orthogonal components (comp [1+4]) as both the values of R² and Q² approach and tend to 1.

The graph at the bottom right shows the software used and the reprocessing date.

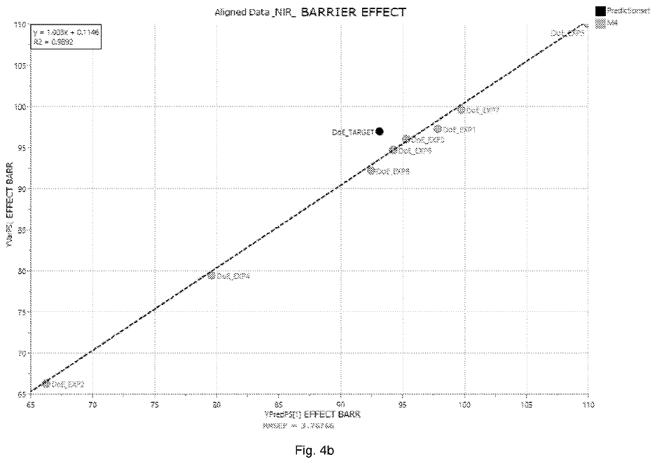

FIG. 4a. Scatter plot obtained using the OPLS algorithm (Orthogonal Partial Least Squares/Orthogonal projections to latent structures, Orthogonal partial least squares/Orthogonal projections to latent structures) of NIR fingerprint data (Near InfraRed) integrated with the barrier effect measurement values (BARR).

The information of the block as predicted (or calculated) data on the abscissa (x-axis). Each circle shown in the graph represents the experimental measurement of the barrier effect of each sample analyzed (see table 7) and characterized by a different composition as described in the text (see table 6). The samples are identified by the acronym “DOE_EXP from 1 to 8” and correspond to the samples called “Experiment from 1 to 8” in table 7.

The dotted line in the center of the graph represents the relationship between the data and is characterized by the equation y=1x+9.003e10⁻⁵, with correlation coefficient R² equal to 0.9991, shown in the graph at the top left.

This straight line expresses a low error value of the estimate, RMSEE equal to 1.07321 (Root Mean Square Error of Estimation, Root of the root mean square error of the estimate), and cross-validation error, RMSE cv equal to 2.14445 (Root Mean Square Error of Cross Validation), which will result in a very accurate prediction of biophysical activity values (Y data block) starting from the NIR fingerprint (X data block). The RMSEE and RMSE cv values are reported under the wording YPRED[1](BARR EFFECT) on the x-axis (x-axis). The vertical bar on the right expresses in color gradient the experimental values of the Y block used to create the model, in this case the measurement values of the barrier effect. The graph at the bottom right shows the software used and the reprocessing date.

FIG. 4b. Scatter plot obtained using the OPLS algorithm (Orthogonal Partial Least Squares/Orthogonal projections to latent structures, Orthogonal partial least squares/Orthogonal projections to latent structures) of NIR fingerprint data (Near InfraRed) integrated with the barrier effect values (BARR) with prediction of the TARGET barrier effect starting from the NIR fingerprint.

The graph is essentially that of FIG. 4a where in addition the TARGET sample (DoE_Target) is present in prediction. Therefore all the details described in the previous graph remain valid. Furthermore, in this case the NIR fingerprint of the TARGET sample has been added to the information of block X, consisting of the NIR fingerprint.

The data of the samples used to build the model are represented by a light gray circle, while the TARGET sample in the prediction model is represented by a dark gray circle. By exploiting the regression model, the fingerprint information of the TARGET sample is used to predict the information of the Y block, in this case to predict the barrier effect information.

The dotted line in the center of the graph represents the interpolation between the data including the TARGET sample and is characterized by the equation y=1.003x+0.1146, with correlation coefficient R² equal to 0.9892, shown in the graph at the top left.

This line expresses a low prediction error value, with the RMSEP equal to 3.76766 (Root Mean Square Error of prediction, root mean square error of prediction), which indicates a very accurate prediction of biophysical activity values.

The value of RMSEP is reported under the wording YPredPS [1](BARR EFFECT) on the x-axis (x-axis).

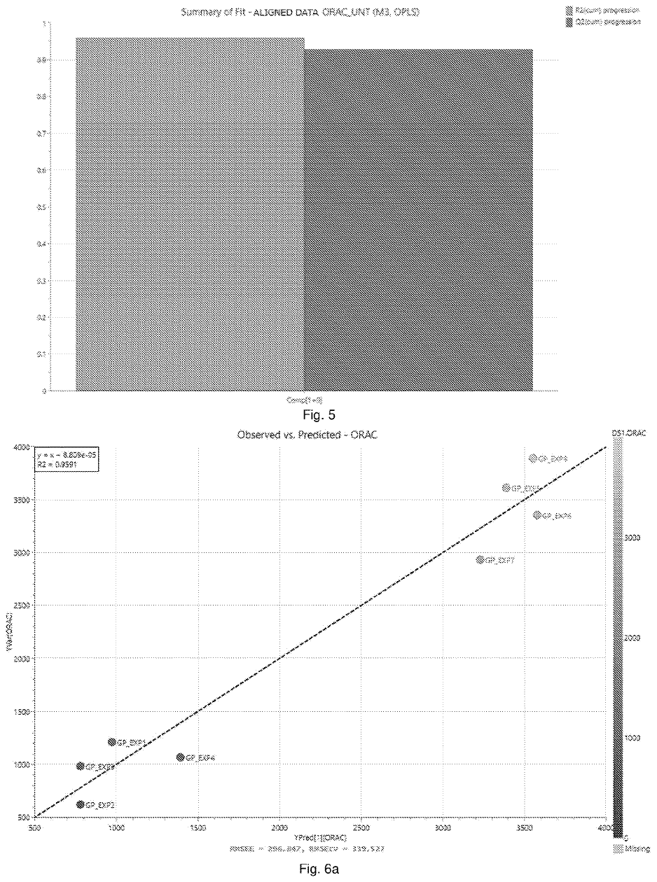

FIG. 5. R² and Q² of the OPLS model with UHPLC-qToF profile (fingerprint Ultra High Performance Liquid Chromatography-quadrupole Time of Flight, fingerprint or profile by Ultra High Performance Liquid Chromatography-quadrupole Time of Flight) & Antioxidant effect.

The OPLS algorithm (Orthogonal Partial Least Squares/Orthogonal projections to latent structures, Orthogonal partial least squares/Orthogonal projections to latent structures) separates the systematic variation of the information better as it separates orthogonal information from parallel information and takes into consideration only the predictive or parallel information.

The information of block X is given by the UHPLC-qToF fingerprint (UNT), while the information of block Y is given by the antioxidant effect (ORAC).

This figure shows the bar graph of the cumulative diagnostic parameters (summary of fit) R² and Q² (in light gray and dark grey, respectively) which express the performance of the OPLS model (model performance) of correlation between the UHPLC-qToF fingerprint and antioxidant effect. R² is a measure that expresses the goodness of the model (the goodness of fit) while Q² is a measure that expresses the goodness of the prediction (the goodness of prediction). The test of the diagnostic parameters R² and Q² is considered satisfied the closer both parameters are to each other and tend towards 1 (y axis). The number of components used is reported on the abscissa (x-axis) and is indicated in square brackets. The two bars represent the value of R² and Q² for the parallel component and no orthogonal component (comp [1+0]).

In this case, therefore, since the value of R² and Q² is very high and close to 1 for the parallel component only, no orthogonal component was used. So, the best OPLS model is the one that uses 1 parallel component and no orthogonal component (comp [1+0]). The graph at the bottom right shows the software used and the reprocessing date.

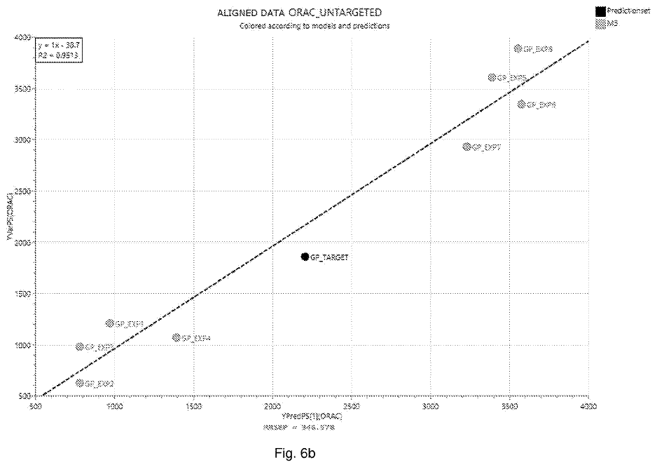

FIG. 6a. Scatter plot obtained using the OPLS algorithm (Orthogonal Partial Least Squares/Orthogonal projections to latent structures, Orthogonal partial least squares/Orthogonal projections to latent structures) of the fingerprint data UHPLC-qToF (Ultra High Performance Liquid Chromatography-quadrupole Time of Flight fingerprint) integrated with antioxidant effect (ORAC) measurement values.

The information of the block) and as predicted (or calculated) data on the abscissa (x-axis). Each circle shown in the graph represents the experimental measurement of the antioxidant effect of each sample analyzed (see table 7) and characterized by a different composition as described in the text (see table 6). The samples are identified by the acronym “DOE_EXP from 1 to 8” and correspond to the samples called “Experiment from 1 to 8” in table 7.

The dotted line in the center of the graph represents the relationship between the data and is characterized by the equation y=1x+8.809e10⁻⁵, with a correlation coefficient R² equal to 0.9591, shown in the graph at the top left.

This straight line expresses a low error value of the estimate, RMSEE equal to 296.847 (Root Mean Square Error of Estimation, Root of the mean squared error of the estimate), and of cross-validation error, RMSE_{CV} equal to 339.527 (Root Mean Square Error of Cross Validation), which will result in a very accurate prediction of biophysical activity values (Y data block) starting from the UHPLC-qToF fingerprint (X data block).

The RMSEE and RMSE_{CV values} are reported under the heading YPRED[1](ORAC) on the x-axis (x-axis). The vertical bar on the right expresses in color gradient the experimental values of the Y block used to create the model, in this case the antioxidant activity values (ORAC values). The graph at the bottom right shows the software used and the reprocessing date.

FIG. 6b. Scatter plot obtained using the OPLS algorithm (Orthogonal Partial Least Squares/Orthogonal projections to latent structures, Orthogonal partial least squares/Orthogonal projections to latent structures) of UHPLC-qToF fingerprint data (Ultra High Performance Liquid Chromatography fingerprint-quadrupole Time of Flight) integrated with the measurement values of the antioxidant effect (ORAC), with prediction of the antioxidant effect of TARGET starting from the UHPLC-qToF fingerprint.

The graph is essentially that of FIG. 6a where in addition the TARGET sample (DoE_Target) is present in prediction. Therefore all the details described in the previous graph remain valid. Furthermore, in this case the UHPLC-qToF fingerprint of the TARGET sample was added to the information of the X block, consisting of the UHPLC-qToF fingerprint.

The data of the samples used to build the model are represented by a light gray circle, while the TARGET sample in the prediction model is represented by a dark gray circle. By exploiting the regression model, the fingerprint information of the TARGET sample is used to predict the information of the Y block, in this case to predict the antioxidant effect information.

The dotted line in the center of the graph represents the interpolation between the data including the TARGET sample and is characterized by the equation y=1x−38.7, with correlation coefficient R² equal to 0.9513, shown in the graph at the top left.

This line expresses a low prediction error value, with the RMSEP equal to 346.658 (Root Mean Square Error of prediction, root mean square error of prediction), which indicates a very accurate prediction of biophysical activity values.

The RMSEP value is reported under YPredPS [1](ORAC) on the x-axis.

### DETAILED DESCRIPTION

The present invention provides a process for the characterization of a product having therapeutic and/or health-promoting properties, said product comprising (or consisting of) complex natural systems, comprising the following steps:
a. carry out a qualitative-quantitative fingerprint analysis of a standard formulation of said product and of formulations that differ from said standard formulation due to the presence of the same ingredients of the same quality at different percentages, or due to the presence of the same ingredients of different quality at the same percentages, thus obtaining an X block of fingerprint data
b. carry out an analysis of one or more biophysical properties of a standard formulation of said product and of formulations that differ from said standard formulation due to the presence of the same ingredients of the same quality at different percentages, or by the presence of the same ingredients of different quality at same percentages, thus obtaining a block of data of biophysical properties
c. carry out an integration by correlation of the data by means of multivariate analysis which involves the correlation of said data block X with said data block Y, integrating by correlation each of the data obtained in a. with each of the data obtained in b, thus obtaining a regression line for each pair of values for which the regression coefficient R² and the mean squared estimate error RMSEE are calculated, selecting those pairs of data wherein R²≥0.8 thus providing a model that allows predict each biophysical property of the standard formulation starting from the fingerprint used.

In this way the regression model allows monitoring the quality of the product not only from the point of view of the qualitative-quantitative fingerprint but also from the point of view of the required biophysical activity and this represents an advancement never achieved until now for the evaluation of the quality of complex natural products.

The data obtained in c. can be used to classify the product as “of compliant quality” or “of non-compliant quality” according to specific product criteria. Compliance criteria are variable and dependent on the specific product.

The present invention also provides a process for the production validation of one or more batches of a product having therapeutic and/or healthy properties comprising complex natural systems comprising the following steps:
a. carry out a qualitative-quantitative fingerprint analysis of a standard formulation of said product and of formulations that differ from said standard formulation due to the presence of the same ingredients of the same quality at different percentages, or due to the presence of the same ingredients of different quality at the same percentages, thus obtaining a block
b. carry out an analysis of one or more biophysical properties of a standard formulation of said product and of formulations that differ from said standard formulation due to the presence of the same ingredients of the same quality at different percentages, or by the presence of the same ingredients of different quality at same percentages, thus obtaining a block Y of biophysical property data;
c. carry out an integration by correlation of the data by means of multivariate analysis which involves the correlation of said data block X with said data block Y, integrating by correlation each of the data obtained in a. with each of the data obtained in b, thus obtaining a regression line for each pair of values for which the regression coefficient R² and the mean squared estimate error RMSEE are calculated, selecting those pairs of data wherein R²≥0.8 thus providing a model that allows predict each biophysical property of the standard formulation starting from the fingerprint used,
d. carry out a qualitative-quantitative fingerprint analysis with the same methods used in a. of one or more batches of said product to be validated, using the model developed in c. so as to predict the biophysical characteristics described in point b.

The data obtained in d. it can therefore be used to classify the product as “of compliant quality” or “of non-compliant quality” according to specific product criteria. The compliance criteria are variable and dependent on the specific product, aimed at maintaining the qualitative-quantitative and biophysical activity parameters.

For each specific product, the acceptance criteria are constructed thanks to fingerprint and biophysical measurements performed on products that differ from the standard formulation. These values represent the conformity limit for each characteristic, beyond which it is necessary to investigate the non-compliant batch.

The availability of this analytical platform allows us to monitor the qualitative-quantitative and biophysical activity parameters of the various production batches and to discard those batches that deviate in such a way as not to guarantee the maintenance of biophysical activity within ranges established a priori, on the basis of ad hoc control charts.

The acquisition of this information on many batches allows us to define very stringent quality ranges by taking into consideration multiple aspects according to a correlation model: the qualitative-quantitative fingerprint and the biophysical activity. Quality ranges can thus be specifically defined depending on the product of interest. In this way the regression model allows monitoring the quality of the product not only from the point of view of the qualitative-quantitative fingerprint but also from the point of view of the biophysical activity and this represents an advancement never achieved to date for quality evaluation of complex natural products.

According to the present invention, said product having therapeutic properties comprising complex natural systems is preferably a product consisting of natural substances, such as for example a medical device according to EC Regulation 745/2017, requirement 13.3 Annex I and/or a food supplement having healthy activities consisting from complex natural substances as defined by Directive 2002/46/EC, article 2 and Legislative Decree 169/2004 article 2.

Examples of such products can therefore be a medical device or a food supplement as defined above, obtainable by mixing natural substances and/or their extracts and/or fractions of said extracts. Preferably the components used for the preparation of said medical device or said food supplement are obtained through physical production processes with low environmental impact.

A non-limiting example of natural substances according to the present invention can be parts of plants, powders of such parts, extracts of plants or their parts (leaves, flowers, roots, bark, stem, seeds, fruits, petioles, parts of flowers etc.), fractions of said extracts (such as hydroalcoholic or other fractions), essential oils, natural gums, natural resins, minerals, honey, propolis, animal substances, mineral substances, etc.

This product can be in different forms such as, for example, in the form of syrup, tablet, capsule, tablet, granules, powder, solution, suspension, aerosol, aerosol powders, powder in sachet, granules in sachet, operculum, spray, cream, emulsion, soft or hard gelatin capsule or other suitable formulations commonly used in the industry.

According to the invention, the formulations that differ from the standard product due to the presence of the same ingredients of the same quality at different percentages, or by the presence of the same ingredients of different quality at the same percentages compared to said sample, are formulations that represent products of poor quality. These formulations can be created by varying the fundamental ingredients for the therapeutic or healthy activity of the product.

To rationally study the contribution of each ingredient on the properties of the complex formulated product, a suitable number of formulas must be created such that the impact on the properties of the formulated product can be assessed. It is therefore necessary to use a suitable experimental design (DOE) in order to discover the conditions of the ingredients that influence the responses.

For classical pharmaceutical products, containing one or two specific active principles (compounds), “full factorial” DOE is used, which involves the creation of 2K formulations, where K=number of active ingredients. In the case of complex natural products, whose components that contribute to the therapeutic effects are multiple, the number of poor quality formulations at point b. they are countless.

For example, in the case of a product made up of 11 complex natural ingredients, more than 1000 formulations would need to be made to describe all the non-quality space around the standard formulation.

Through preliminary studies, the conditions of the ingredients necessary to maintain the characteristics of the product can therefore be identified. This can be done by using quality (or standard) samples, which represent the target (or central point of an experimental design), and poor quality samples, which differ from the quality samples by the presence of the same ingredients of equal quality at percentages different, or due to the presence of the same ingredients of different quality at the same percentages compared to the target.

To select poor quality formulations, the ingredients of the product of interest can be organized into groups.

In an embodiment of the invention, poor quality formulations can be selected by organizing the ingredients into groups where there are 3 fundamental groups which, by varying, modify the properties of the formulation (in table 4 in the examples section, these groups are defined as “factors” and are A, B, C), and a further group can be identified with all the other ingredients of the product definable as an independent factorial group in table 4, these ingredients are in the group defined as factor D). Using a “computer aided” experimental design with a non-orthogonal factorial design, the different percentages of grouped ingredients (factors A, B, C) can be made to vary in random combination and the factor D is combined by complement to 100. Through this calculation process it is possible to select the formulations of “poor quality suitable for generating a regression line” i.e. those that will have the highest probability of constructing a line to be used as a model to predict new data. For the product analyzed in the examples section, 8 formulations were chosen, shown in table 6.

With respect to a “full factorial” type DOE study, the number of poor quality formulations to be studied is therefore significantly reduced.

The qualitative-quantitative fingerprint analysis in point a. can be carried out using known techniques capable of describing the complexity of the product of interest: such as, for example, infrared (IR) and near infrared (NIR) spectroscopy, mass spectrometry (MS, and various possible couplings), nuclear magnetic resonance (NMR), ultraviolet-visible spectroscopy (UV-Vis). According to one embodiment, said fingerprint analysis is carried out by UHPLC-qToF, recording of the NIR spectrum, recording of the IR spectrum, or by acquisition of chromatograms with other mass spectrometers (ion trap, triple quadrupole, single quadrupole) also coupled to different chromatographic systems (for example: gas chromatography, liquid chromatography), or by direct infusion into mass spectrometers, NMR, UV-Vis spectroscopy also coupled to chromatographic systems (for example: liquid chromatography).

According to the present invention, at point b. one or more biophysical properties correlated with the therapeutic efficacy of the product of interest are analysed. A non-limiting example of such properties is represented by the mucoadhesion property, the barrier effect, the antioxidant, neutralizing, hydrating, lubricating properties, etc.

Therefore, in one embodiment of the invention, in point b. said analysis of the biophysical properties can be carried out by means of one or more of: measurement of the mucoadhesion capacity, measurement of the barrier effect, measurement of the antioxidant effect, measurement of the neutralizing activity, measurement of the hydrating activity, measurement of the lubricating activity.

The analysis of these properties can be carried out using any methodology known to the expert in the sector, a non-limiting example of such technologies includes for example the measurement of the (mucus) adhesion effect, through tests on an inclined plane, the measurement of the barrier effect, via in vitro tests on human fibroblast cells or via tests to reduce the passage of the Dextran-Fluorescein molecule (70,000 MW) on a semi-permeable membrane (transwell), and the measurement of the antioxidant effect, via ORAC tests.

In a preferred embodiment the integration between the data at point c. is carried out using the OPLS algorithm.

In OPLS, data is separated into predictive (or parallel) information and uncorrelated (or orthogonal) information, thus removing variation from X (descriptive variable block) that is not related to Y (property variable block) generating a highly predictive correlation model. Therefore the OPLS model is better as it separates the orthogonal information from the parallel information and takes into consideration only the predictive or parallel information.

Algorithms or functions suitable for processing NIR fingerprint data by scaling are for example the SNV (Standard Normal Variate) algorithm, first derivative and second derivative; for the fingerprint by mass spectrometry the PARETO and MEAN-CENTERING algorithms; for biophysical data the UV (Unit Variance) or CENTERING algorithm well known to the technician in the sector.

Algorithms or functions suitable for processing the data relating to the analysis of each biophysical property by transformation are for example the EXPONENTIAL function (for example squared exponent, ¼ exponent), or the LOGARITHM function.

The processed variables can be evaluated by calculating the diagnostic parameters of the model R² and Q². Data pairs that have an adequate R² are selected, such as R²≥0.8, preferably R²≥0.9, preferably ≥0.95, even preferably greater than or equal to 0.99. In any part of this description the term “including” may be replaced by the term “consisting of”.

The following examples were created on standard formulations of syrup A (Grintuss produced by Aboca) as defined below and on formulations that differ from said standard formulation due to the presence of the same ingredients of equal quality at different percentages, or due to the presence of the same ingredients of different quality at the same percentages. The examples provided are intended to illustrate possible ways of carrying out the invention or some of the steps of the claimed processes and are not to be understood in any way as limiting the same.

### EXAMPLES

### Methods Used

### 1. Method for Determining the NIR Fingerprint-Reflection Mode

### 1.1. Instrumentation

Bruker NIR spectrometers, model MPA (Multi Purpose Analyzer)

### 1.2. Parameters of the Reflection Method.

Resolution: 16 cm⁻¹; Wavenumber Reproducibility: Better than 0.04 cm⁻¹; Wavenumber
Accuracy: Better than 0.1 cm⁻¹; Photometric Accuracy: 0.1% T; Wavenumber range: from 4000 to 12000 cm⁻¹; Background scans: 64; Scans for sample acquisition: 64

### 1.3. Sample Preparation

For NIR analysis, 3 ml of each syrup sample was deposited in the center of a Petri dish by using a disposable plastic pipette. A metal spacer was positioned above the drop, taking care to eliminate any air bubbles that could form between the spacer and the plate.

### 1.4. Sample Acquisition

For each sample, three technical replicates were acquired, repeating the acquisition three times to be able to monitor the reproducibility of the data. Background subtraction was performed before each acquisition.

### 1.5. Data Pre-Processing

For the development of the reprocessing method, for which the evaluation of the pre-processing of the data is also necessary, the SIMCA® software was used thanks to which it was possible to choose as the pre-processing method the one which involves the use of SNV normalization.

### 1.6. Data Evaluation Using SIMCA®

A new sample introduced into a NIR model is evaluated through statistical tests that demonstrate their belonging to a range of variance for which the model is validated. The aforementioned diagnostic tests are Hotellings T² and DModX.

### 2. Method for Determining the NIR Fingerprint-Transmission Mode

This method differs from the previous one in the way wherein the sample is presented for instrumental reading, which in this case involves the use of a test tube with an optical path of 2 mm.

### 3. Method for Determining the UHPLC-qToF Fingerprint

### 3.1. Reagents

Methanol/Ultrapure Water 50:50 (v/v), HPLC Grade Methanol (MeOH), Ultrapure Water

### 3.2. Materials

Column: Waters Cortecs® C18 1.6 μm 2.1×100 mm; Precolumn: Waters Cortecs C18 1.6 μm 2.1×5 mm VanGuard™ Pre-Column 3/Pk; Syringe filters, 0.2 μm RC Membrane, Phenomenex

### 3.3. Instrumentation

UHPLC-qToF System: Agilent 1290 Infinity coupled to a 6545-quadrupole time-of-flight (qToF) with 2 GHz resolution with Electrospray Dual AJS ESI source (Agilent Technologies). Ultrasonic bath. Analytical balance (resolution 0.00001 g).

### 3.4. Sample Preparation

Using a glass pasteur, weigh 0.500 g±0.005 g of sample into a 50 mL volumetric flask. Using ultrasound, solubilize the sample for 15 min at a temperature of 35° C.±3° C. with approximately 45 mL of 50% MeOH for LC-MS. At room temperature, make up to 50 ml with the same extraction solvent. Filter 3 ml of the extract through a 0.2 μm cellulose acetate filter into a 4 mL amber vial, discarding the first 0.5 ml of filtrate. Take 200 μL of filtered sample with a calibrated 200 μL micropipette and transfer them into an amber vial with glass insert and screw cap. Inject into UHPLC-qToF system in negative polarity [ESI (−)] for the determination of the metabolomic profile.

### 3.5. UHPLC Chromatographic Conditions

Injection volume: 3 μl; Column temperature: 40° C. Eluents: A. H2O, 0.1% HCOOH: B. MeOH, 0.1% HCOOH

Time segment: 0 Waste; 2 min MS; Flow: 0.3 mL/min. Elution scheme:

| Time (min) | A (%) | B (%) |
|---|---|---|
| 0 | 99 | 1 |
| 1 | 99 | 1 |
| 2 | 75 | 25 |
| 10 | 50 | 50 |
| 11 | 50 | 50 |
| 15 | 25 | 75 |
| 17 | 15 | 85 |
| 19 | 1 | 99 |
| 19.5 | 1 | 99 |
| 21 | 99 | 1 |

Post-time: 3 min; Iso pump: 0.05 mL/min

### 3.6. Reprocessing of Fingerprint Data Obtained Via UHPLC-qToF

As a first step, the features extraction (data.extraction) is performed using the XCMS package (version 3.2.0) which uses the following algorithm:
centWave

The algorithm performs operations such as peak picking and alignment. The parameters shown in table no 2 were used to execute the algorithm.

TABLE NO. 2
Definition of parameters set for data.extraction function execution

1st step
Function data.extraction

| | | Parameter | |
|---|---|---|---|
| Name | Description | used | |
| PPM | Represents the accepted error in | 10 | ppm |
| | experimental mz value compared to | | |
| | the theoretical value (mass | | |
| | accuracy) | | |
| MIN | Represents the minimum amplitude | 10 | s |
| | in seconds to define a peak. | | |
| MAX | Represents the maximum amplitude | 45 | s |
| | in seconds to define a peak. | | |
| k1 | Represents the prefilter parameter, | 3 | scans |
| | i.e. the minimum number of scans to | | |
| | define a peak. | | |
| t1 | Represents the value of prefilter | 500 | count |
| | parameter, i.e. the minimum height | | |
| | to define a peak. | | |
| SN | It represents the signal/noise ratio | 10 | |
| | adequate for the technical- | | |
| | experimental conditions. | | |
| filling | Applies (y) or does not apply (n) | y | |
| | peak filling for missing imputation | | |
| | value (missing data). | | |
| resM | Represents the resolution in ppm to | 10 | ppm |
| | carry out the peak grouping. | | |
| restT | represents the retention time | 15 | s |

| | | |
|---|---|---|
| | resolution to carry out the peak | |
| | grouping. | |
| Polarity | Ionization mode used: negative | negative |
| | (negative) or positive (positive) | |

As a second step, a filtering function of the obtained variables called pre-processing is used.

The parameters shown in table no 3 were used to execute the function.

TABLE NO. 3
Definition of parameters to be set for
execution of the preprocessing function

| | 2nd step | |
|---|---|---|
| | Preprocessing function | |
| | | Parameter |
| Name | Description | used |
| xNA | Sets the missing data filter threshold for | 0.2 |
| (NAlimit) | variables in Pools expressed as a fraction. | |
| CVmax | Set the threshold for the filter on the CV of the | 0.2 |
| (CVlimit) | variables in the Pools expressed as a fraction. | |
| | The coefficient of variation (CV) is an index of | |
| | dispersion that allows us to evaluate the | |
| | dispersion of values around an average value. | |
| PQN | Apply (y) or do not apply (n) normalization | n |
| (normalize) | using PQN (Probabilistic Quotient Normaliza- | |
| | tion) using the “Pool” sample variables | |
| | as a basis. | |

The last step of variable extraction consists in subtracting the Analytical Blank. It is done through the blanksubtract.centWave function. (table no 4).

TABLE NO. 4
Definition of parameters to be set for execution
of the blanksubtract.centWave function

| | 3rd step | | |
|---|---|---|---|
| | blanksubtract.centWave | | |
| | | Parameter | |
| Name | Description | used | |
| dataset | The data matrix obtained by running the peak | — | |
| | picking algorithm. | | |
| PPM | Represents the accepted error in experimental | 10 | ppm |
| | mz value compared to the theoretical value | | |
| | (mass accuracy). | | |
| MIN | Represents the minimum amplitude in | 10 | s* |
| | seconds to define a peak. | | |
| MAX | Represents the maximum amplitude in | 45 | s* |
| | seconds to define a peak. | | |
| k1 | Represents the prefilter parameter, i.e. the | 3 | scans |
| | minimum number of scans to define a peak. | | |
| t1 | Represents the value of prefilter parameter, | 500 | counts * |
| | i.e. the minimum height to define a peak. | | |
| SN | It represents the signal/noise ratio adequate | 10 | |
| | for the technical-experimental conditions. | | |
| resM | Represents the resolution in ppm for the | 10 | ppm |
| | identification of equivalent variables between | | |
| | datasets and “BiancoA” based on m/z. | | |
| restT | R represents the resolution in seconds for the | 10 | s |
| | identification of equivalent variables between | | |
| | datasets and “BiancoA” based on retention | | |
| | times. | | |
| M | Represents the weight parameter for the | 0.5 | |
| | desirability function in the evaluation of the | | |
| | equivalence of the variables based on mz and | | |
| | Rt. Parameter between 0 and 1. 0.5 gives | | |
| | equal weight to resM and resT | | |
| alpha | Represents the percentile value for excluding | 0.1 | |
| | common variables. | | |

*optimized with IPO using the following ranges: t1 = 500, 5 < MIN > 20, 30 < MAX > 60

The final data matrix thus obtained is converted into a .csv file loaded into the SIMCA® software in order to proceed with the statistical reprocessing using OPLS.

### 4. Method for Determining the Adhesion Activity on an Inclined Plane

Prepare a reference standard solution (RSS) of 0.9% w/w sodium alginate in physiological saline. Analyze the sample to be tested (SAMPLE) (syrup, SYRUP A) without any manipulation.

Before starting the analysis, ensure that the RSS and SAMPLE samples have a temperature of 20° C.

Using a micropipette, deposit an equivalent number of drops of SAMPLE and RSS at the beginning of a plate with a defined slope. Deposit the drops on the same line, equidistant from each other. Due to the effect of gravity, the sample and RSS move along the plate. The SAMPLE test is compliant if it has a higher adhesion capacity than the RSS.

### 5. Method for Determining the Barrier Effect

The assay is based on the principle whereby the reduction in the passage of the Dextran-Fluorescein molecule (70,000 MW) after the application of the product is considered an index of the protective efficacy of the formulation compared to an untreated control (no formulation applied, represents the positive control).

The test requires the setup of two chambers physically separated by a semi-permeable membrane (transwell). The product is applied to the surface of the transwell and after 10 minutes the Dextran-Fluorescein is applied. The solution in the receptor is collected one hour after the application of Dextran and is read on a plate reader by setting 494 nm of excitation and 518 nm of emission, considering that the barrier effect can be determined by measuring the fluorescence of the well solution of the receiving plate. A white is also considered (application of the product without dextran). This condition is used to evaluate the contribution of a potentially diffuse portion of the product inside the receiving chamber.

After calculating the quantity of fluorescein dextran present in the sample and in the positive control, the percentage of the barrier effect is calculated using the following formula:

% barrier effect=100−((sample dextran concentration/average positive control dextran concentration))*100

### Reagents and Instrumentation

Dextran Fluorescein D1823 70,000 MW (Invitrogen)
PBS (Gibco)
HTS 96 3391-Corning® HTS Transwell®
Corning 3605-96 round bottom white plate+lid
Fluorescence reader for plates (Varioskan-Thermo Fisher)
Eppendorf 1.5 ml
Thermo Fisher p10, p20, p100, p1000 pipettes+tips
Multipette R Xstream Eppendorf+5 ml tips
Falcon 15 and 50 ml
Vortex
Timer

### Execution of the Assay

The reagents needed to perform the test are:
Dextran Fluorescein: it is necessary to resuspend the powder with PBS at a concentration of 25 mg/ml as indicated on the datasheet. This represents the stock and it is necessary to make aliquots and store them at −20° C. (vortex the stock solution before aliquoting). The concentration of use in the test is 500 μg/ml, therefore it must be diluted 1:50 in PBS.
sample: the sample must be mixed before starting the test and transferred approximately 5 ml into a 15 ml falcon.
PBS: aliquot the required amount of PBS into a 50 ml bottle. PBS is stored at room temperature.

Below are the experimental set and the steps for carrying out the test. Each sample must be in triplicate.
Positive control (CTRL+): 15 μl of PBS top, 90 μl of PBS bottom, 2 μl of Dextran Fluorescein at a concentration of 500 μg/ml top
sample: 15 μl sample top, 90 μl PBS bottom, 2 μl Dextran Fluorescein at a concentration of 500 μg/m top

Blank: 15 μl of sample in the top, 90 μl of PBS in the bottom, 2 μl of PBS in the top. This condition is used to evaluate the contribution of a potentially widespread portion of the product inside the receiving chamber.
1. Mix the sample to be tested
2. Prepare 500 μg/ml Dextran solution Fluorescein and keep in the dark
3. Add 15 μl of syrup (for sample and blank) or 15 μl of PBS (for ctrl+) on the surface of the HTS 96 transwell and leave 10′ RT. Change tip for each transwell.
4. At the end of 10′, add 90 μl of PBS to the bottom+vortex the 500 μg/ml Dextran solution for the first time Fluorescein and add 2 μl to the top for the sample and for the ctrl+. For the blank add 2 μl of PBS to the top.
5. Incubate 1 h RT in the dark
6. Prepare calibration curve in Eppendorf and keep in the dark at RT 1 h
7. Transfer 75 μl (in duplicate) of each curve point and samples into 96 wells U bottom white plates and read the fluorescence with varioskan (Ex 494 nm, Em 521 nm)

### Quantification of Dextran Fluorescein

Once the fluorescence readings have been taken by setting the excitation and emission wavelengths to 494 and 521 nm respectively, generate the dextran calibration curve fluorescein with GRAPHPAD PRISM software. Set the function to subtract the average of the fluorescence values of the blank wells from the fluorescence values of the sample. Then calculate the concentration of dextran fluorescein present in the sample and in the positive control by interpolating the fluorescence values of the corresponding wells with the standard curve.

### Analysis of the Results

After calculating the amount of dextran fluorescein present in the sample and in the positive control, calculate the percentage of barrier effect using the following formula:

$$\% barrier effect = 100 - ( ( sample Dextran concentration / average Dextran concentration ctrl + ) ) * 100$$

The reduction of the passage of Dextran Fluorescein following the application of the product is considered an index of the protective efficacy of the formulation compared to an untreated control (no formulation applied).

### 6. Method for the Determination of Antioxidant Properties by ORAC Measurement (Oxygen Radical Absorbance/Antioxydant Capacity)

The method was developed with the aim of evaluating the protection that antioxidant substances provide to the organism against reactive hydroxides and peroxides. The method is designed to test human and animal serum samples, plant products, foods, food ingredients, pharmaceuticals and pet foods. At the moment, it is considered the only method capable of measuring the inhibitory capacity that an antioxidant can exert on free radicals. The unit of measurement of antioxidant power has been given the name ORAC unit.

This is a very sensitive method that uses Fluorescein as a fluorescence marker and 2,2′-azobis(2-amindinopropane) dihydrochloride (AAPH), a water-soluble azo compound that decomposes thermally, leading to the formation of aqueous peroxy radicals at constant speed. Fluorescein fluorescence is highly sensitive to the conformation and chemical integrity of the molecule itself. Under appropriate conditions, the loss of fluorescence in the presence of peroxy radicals is an indicator of the oxidative damage generated by the reactive species. In a given sample, the inhibition of the degradation of Fluorescein, which is reflected in the preservation of its fluorescence, thanks to the protective action of the antioxidants present, is a measure of the antioxidant capacity of the sample towards reactive species. As long as antioxidants are able to capture radicals, they protect the fluorescence marker from decay; once the effect of the antioxidants is over, the radicals react with the Fluoresce wherein loses fluorescence. The fluorescence decay time is proportional to the quantity and activity of the antioxidants present in the sample.

The ORAC assay therefore measures the time-dependent decrease in the fluorescence of the marker molecule Fluorescein, as a consequence of the damage caused by the oxygen-focused radical.

The results of the assay are quantified by allowing the reaction to reach completion and subsequently integrating the area under the kinetic curve relative to a blank reaction containing no added antioxidants. The area under the curve (AUC) is proportional to the concentration of all antioxidants present in the sample. This method allows you to combine two parameters, the inhibition time and the percentage inhibition of the reactive species due to the total antioxidants calculated as a single quantity: for this reason the method is better than similar ones which use percentage inhibition at a fixed time or the inhibition time at a fixed percentage of inhibition.

The final results are calculated using the difference in Fluorescein decay AUC (relative fluorescence vs. time) between the test sample and the blank.

Trolox® standard (6-hydroxy-2,5,7,8-tetramethylchroman-2-carboxylic acid) a water-soluble analogue of vitamin E.

$$ORAC UNIT = ( AUC Sample - AUC blank ) / ( AUC std - AUC blank ) \times [ Std ] / [ Sample ] = μ mol / g$$

Assay results are reported based on the equivalence 1 ORAC unit=1 μM Trolox® equivalents per 100 g of sample.

### Example 1 Complex Product Characterization

Grintuss, also reported here as “Syrup A”) against cough has been characterized and produced through a highly standardized production process consisting of a mixture of the following ingredients:
Polyresin based on polysaccharides, resins and flavonoids ((LPME extraction fraction of Plantain, Grindelia and Helichrysum obtained by subjecting the three starting plants (Plantain, Grindelia, Helichrysum) to a physical extraction process with low environmental impact (LPME process, Liquid Phase Micro Extraction, a process characterized by the liquid phase extraction of plant parts reduced into particles of very microscopic dimensions) capable of producing extractive fractions and standardizing their content within the finished product)), honey, sugar cane, water, essential oils of eucalyptus, star anise and lemon, natural lemon flavouring, gum arabic and xanthan. All experiments described below were performed in triplicate.

### 1. Experimental Design (DOE) and Characterization of the Experimental Design Samples

Different quality formulations of the original complex natural product were defined in order to obtain a correlation model that could integrate the qualitative-quantitative characteristics of the product with the corresponding biophysical characteristics. This model used for the characterization of numerous quality samples will allow the definition of stringent quality levels of the same product.
In the case of syrup A, table 5 shows all the ingredients grouped in factors A (this factor is represented by the ingredients Polyresin, Gum Arabic and Xanthan Gum), B (this factor is represented by the ingredient Honey) and C (this factor is represented by the ingredients sugar and water) identified as important for the purposes of studying its effect on the properties of the complex natural product. Factor D is reported as it represents the other ingredients of the formulation (ingredients: water, lemon aroma, lemon and eucalyptus essential oil), but for the purposes of the study it was considered as an independent factor.

TABLE no 5
Composition of the factors of the DOE study

| | | | | | | Natural | | Lemon | Star Anise | Eucalyptus | |
|---|---|---|---|---|---|---|---|---|---|---|---|
| | | | | Arabic | Xanthan | Lemon | | Essential | Essential | Essential | |
| ID | Water | Polyresin | Sugar | gum | gum | Flavor | Honey | Oil | Oil | Oil | Total |
| TO | | x | | x | x | | | | | | 1 |
| B | | | | | | | x | | | | 1 |
| C | x | | x | | | | | | | | 1 |
| D | x | | | | | x | | x | x | x | 1 |

Note 1.
Mixture D was considered as an independent factor

Starting from the composition of the TARGET product (syrup A), applying computer-aided design (CAD), the factors A, B and C were increased or decreased to cover a box of compositions centered on the TARGET generating a grid of possible combinations. Only grid elements that satisfied the formulation constraint were included in the candidate experiment set, which was sampled to select the 8 formulations to be considered for studying the responses. Table 6 therefore shows the combinations of factors accepted with the percentages of increase or decrease compared to the target. In particular, factor A was found to vary in a range of ±80%, while factors B and C were found to vary in a range of ±20%.

The combination procedure carried out by computer aimed to obtain a DOE matrix with a “condition number” minimum. Therefore, thanks to the formulative experimental design procedure, the distance between the candidate formulations and the experiments of a complete factorial design has been reduced to a minimum, therefore to 2³ (2^{K}) since three factors (A, B, C) are considered, equal to 8 experiments.

Table 6 therefore reports the formulations to be investigated. The above DOE matrix supports linear models and interaction models with a “condition number” less than 3.6.

TABLE NO. 6
Variation of the percentages compared to the target of the AC
factors in the experimental samples used in the DOE study

| ID | A % | B % | C % | D % |
|---|---|---|---|---|
| Experiment 1 | −72 | −8 | −4 | 100's |
| Experiment 2 | −80 | −20 | +16 | complement |
| Experiment 3 | −80 | +20 | −12 | to the target |
| Experiment 4 | −56 | +16 | +4 | formula |
| Experiment 5 | +80 | −18 | −4 | |
| Experiment 6 | +80 | −20 | +12 | |
| Experiment 7 | +40 | +20 | −16 | |
| Experiment 8 | +72 | +8 | +4 | |

The formulations called experiment 1 to 8 were used for recording the fingerprint and biophysical properties.

### 1.1—Fingerprint Using NIR Spectroscopy.

The NIR field from 4000 to 12500 cm⁻¹ was selected and the background noise was subtracted. The recording of the spectrum can be carried out in transmission or reflection mode, where in optical spectroscopy the transmission T is the ratio between the intensity of a transmitted light ray (lₜ) and the incident light ray (l₀), while the reflection is the ratio between the intensity of a reflected light ray (lᵣ) and the incident light ray (l₀).

The NIR spectrum is transformed into a data matrix with the variables characterized by the wavelength and the corresponding transmittance or reflectance value. The aforementioned matrix with the data of all the samples studied is aligned and pre-processed, i.e. transformed using the SNV (Standard Normal Variate) algorithm and scaled using the MEAN-CENTERING function. Further pre-processing functions can be the first derivative and the second derivative, or combinations of these with the SNV algorithm. The final data matrix thus obtained is used in the next phase of integration with the biophysical data using OPLS.

The software packages that can be used to carry out the analysis of NIR spectra are those proprietary to the NIR instrument, for example for the Bruker uses the OPUS software.

Another software used for NIR spectra analysis is SIMCA® (Sartorius)

### 1.2—Fingerprint by UHPLC-qToF Mass Spectrometry.

The extraction of the features (these are the variables) has been carried out from the fingerprint was performed using the peak picking and peak operations alignment. The parameters shown in table no 2 were used to execute the algorithm.

As a second step, a filtering function was used for the variables obtained using the parameters reported in table no 3. The last step of the extraction of the variables consists in the subtraction of the analytical blank (table no 4). The final data matrix thus obtained is converted into a .csv file and used for the subsequent phase of integration with the activity data to proceed with the statistical reprocessing using OPLS correlation. As an example of software packages that can be used to carry out data extraction, data processing and data analysis, XCMS and SIMCA® (Sartorius) can be used.

### 1.3—Measurement of Adhesion Properties

This biophysical property was measured by placing the complex natural product (e.g. Syrup A) on an inclined plane which moves by gravity. The tested product must be more adhesive than the reference and this is verified by measuring the mm traveled by the product and the reference on the inclined plane.

The measurement is made in conditions of constant temperature and with the inclined plane with a defined and fixed angle, in order to make the measurement robust and repeatable.

The detailed procedure used in this context is given as an example in the materials and methods section.

### 1.4—Measurement of Barrier Properties

This biophysical property was evaluated by placing the product on a semi-permeable membrane (transwell) and measuring its ability to prevent the passage of the compound dextran-fluorescin (70000 Molecular weight).

The detailed procedure used in this context is given as an example in the materials and methods section.

### 1.5—Measurement of Antioxidant Properties

This property was measured using the ORAC test, according to standard protocols known to those skilled in the art. The detailed procedure used in this context is given as an example in the materials and methods section.

### 2. Calculation of Multilinear Regression

The experimental measurements of the biophysical properties used for the calculation of the multilinear regression are reported in table no 7.

TABLE NO. 7
Measurements of antioxidant activity, adhesion activity and barrier
effect in the experimental samples used in the DOE study

| | Antioxidant | Adhesive | Barrier |
|---|---|---|---|
| | activity | activities | effect |
| ID2 | (μmol TE/100 g) | (mm run) | (%) |
| Experiment 1 | 1210 | 60.5 | 97.25 |
| Experiment 2 | 623 | 24.33 | 66.3 |
| Experiment 3 | 982 | 53.83 | 96 |
| Experiment 4 | 1070 | 22.33 | 79.44 |
| Experiment 5 | 3610 | 34.17 | >100 110 (*) |
| Experiment 6 | 3350 | 10.67 | 94.66 |
| Experiment 7 | 2930 | 31.17 | 99.56 |
| Experiment 8 | 3890 | 10.33 | 92.16 |

Note 2.
(*) this data was calculated in prediction and will be used to build the models

Before proceeding with the evaluation of the relationship between fingerprint and biophysical response (which will be done using OPLS), an analysis of the biophysical responses was carried out based on multilinear regression, a classic least squares regression, to verify the presence of linearity in a interaction model between the ingredients of the 3 factors A, B, C in the 8 different formulations (experiments from 1 to 8). On the basis of equation 1, the different correlation coefficients were therefore evaluated and the results are reported in table 8.

$$f ( Y ) = aA + bB + cC + dAB + eAC + fBC + g \qquad Equation ( 1 )$$

Where: f (Y) transformed function of the evaluated response
a,b,c=regression coefficients that express the effect of the variation of
Parameters A, B, C on the antioxidant biophysical activity (a), adhesive effect (b), barrier effect (c)
d,e,f=regression coefficients that express the effect of the variation of the combined parameters AB, AC, BC on the antioxidant biophysical activity (a), adhesive effect (b), barrier effect (c)
A, B, C=variable ingredient mixtures in experiments 1-8
g=intercept

The data relating to the biophysical parameters were subjected to transformation (also called Box-Cox transformations; Box, G E P and Cox, DR (1964). An analysis of transformations, Journal of the Royal Statistical Society, Series B, 26, 211-252.) using the logarithm base 10, exponent ¼, exponent squared functions for the antioxidant activity, for the adhesive activity and for the barrier effect, respectively.

Table 8 shows the results of the multiple linear regression evaluation. For the models of the three activities evaluated the values of Q² and R² are good.

TABLE no 8
Evaluation of interaction models using Multiple Linear Regression of biophysical activity data

| | Coeff. | | Adhesive | Coeff. | | Barrier | Coeff. | |
|---|---|---|---|---|---|---|---|---|
| log(Att. antiox) | SC | p-value | effect ^{1/4} | SC | p-value | Eff. ² | SC | p-value |
| g | 3,259 | <0.001 | g | 2,296 | <0.001 | g | 8820 | <0.001 |
| a | 0.317 | <0.001 | a | −0.288 | <0.001 | a | 1130 | 0.047 |
| | | | b | −0.262 | <0.001 | b | −1300 | 0.129 |
| c | −0.085 | 0.014 | c | −0.555 | <0.001 | c | −3300 | 0.0011 |
| | | | f | 0.068 | 0.022 | | | |
| | Q ²= | 0.93 | | Q ²= | 0.98 | | Q ²= | 0.21 |
| | R ²= | 0.97 | | R ²= | 0.99 | | R ²= | 0.78 |
| | ANOVA= | <0.001 | | ANOVA= | <0.001 | | ANOVA= | 0.022 |
| | cn= | 1.81 | | cn= | 2.96 | | cn= | 3.46 |
| | | | | | | | EXP5= | 110 |

Note 3
Coeff. SC stands for scaled coefficient, for logarithm function base 10, exponent ¼ or 0.25 and exponent squared, respectively.

The coefficient a indicates the correlation of the A factor of mixtures 1-8, therefore there is good correlation (directly proportional) with the antioxidant activity and quite good correlation (inversely proportional) with the adhesive effect. Factor A is linked to the presence of Poliresin and gums (xantahan gum and gum arabic).

The coefficient b indicates the correlation of the B factor of mixtures 1-8, so there is quite good correlation (inversely proportional) with the adhesive effect and quite good correlation (inversely proportional) with the barrier effect. Factor B is linked to the presence of honey.

The coefficient c indicates the correlation of the C factor of mixtures 1-8, therefore there is good correlation (directly proportional) with the adhesive effect and quite good correlation (inversely proportional) with the barrier effect. Factor C is linked to the presence of sugar and water.

The coefficient f indicates the correlation of the combined BC factors of mixtures 1-8, therefore there is good correlation (directly proportional) with the adhesive effect. The combined BC factor is linked to the presence of honey, sugar and water.

By inserting the data from table 8 into equation 1, the corresponding regression equation is obtained which made it possible to study the relationships between the responses of factors A, B and C in the experimental formulas 1-8 (table n°7) considering the models of interaction. Box-Cox transformations were applied to the responses to obtain normally distributed residuals.

The ORAC response was base10 logarithm transformed, providing a linear model with the following equation:

$${log}_{10} ( ORAC ) = 3.259 ( p < 0. 001 ) + 0.3 \; 17 ( p < 0. 0 \; 01 ) A - 0.085 ( p = 0. 0 \; 14 ) C$$

The “condition number” is 1.81, and the analysis of variance (ANOVA) p value was less than 0.001. The goodness of fit of the multilinear regression model was R²=0.97. Leave-one-out (loo) cross-validation resulted in a Q²=0.93.

The adhesion response to the inclined plane was transformed to the 0.25 power, providing an interaction model with the following equation:

$${( Adhesion to the inclined plane )}^{0.25} = 2.296 ( p < 0. 001 ) - 0.2 \; 88 ( p < 0. 0 \; 01 ) A - 0.262 ( p < 0. 0 \; 01 ) B - 0.555 ( p < 0. 0 \; 01 ) C + 0.0 \; 68 ( p = 0. 0 \; 22 ) BC$$

The “condition number” is 2.96 and ANOVA resulted in a p value less than 0.001. The model showed an R²=0.99 and a Q²=0.98.

The “Barrier Effect” response was transformed by power squared, providing a linear model with the following equation:

$${( Barrier effect )}^{2} = 8820 ( p < 0. 001 ) - 11 \; 30 ( p = 0. 0 \; 47 ) A - 1300 ( p = 0. 1 \; 29 ) B - 3300 ( p = 0. 0 \; 11 ) C .$$

The value of experiment 5, whose response was found to be saturated, was excluded from the calculation. The “condition number” is 3.46 and the ANOVA test provided a p value of 0.022. The model showed R²=0.78 and Q²=0.21. The response in Experiment 5 was therefore estimated to be 110.

### 3. Integration by OPLS Correlation of Qualitative-Quantitative Data and Biophysical Properties

On the basis of the positive results obtained from the study of the multilinear regression of the biophysical properties measured for mixtures 1-8, we proceeded with the construction of correlation models between qualitative and quantitative fingerprint data and biophysical data through the algorithm of orthogonal projections to structures latent (OPLS). In OPLS, experimental data is separated into predictive (or parallel) information and uncorrelated (orthogonal) information, thus removing variation from X (descriptor variables) that is not related to Y (property variables). Therefore, OPLS models are better because they only take into account the predictive or parallel information, separating it from the non-predictive or orthogonal information.

Similar correlations can also be obtained using the PLS, PLS2 algorithms (for definitions see pages 14-15-16, Mattoli L, Gianni M, Burico M. Mass spectrometry-based metabolomics analysis as a tool for quality control of natural complex products. Mass Spectrom Rev. 2022 Mar. 3: e 21773. doi: 10.1002/mas.21773. Epub ahead of print. PMID: 35238411; See pages 430-431 User Guide to SIMCA 15 By Sartorius (https://www.sartorius.com/download/544940/simca-15-user-guide-en-b-00076-sartorius-data.pdf; See: Stocchero M., Riccadonna S., Franceschi P., Projection to latent structures with orthogonal constraints for metabolomics data, Journal of Chemometrics. 2018; e 2987. https://doi.org/10.1002/cem.2987)

3.1. OPLS Correlation Integration of NIR Fingerprint Data with Adhesion Data

Fingerprint data were pre-processed using the SNV (Standard Normal Variate) algorithm and scaled using the MEAN-CENTERING function. The data relating to the measurement of adhesion activity were transformed using the exponent function ¼ (or 0.25) and scaled using the UV (Unit Variance) function. In general the transformation algorithms can be exponential or logarithmic such as: Log, NegLog, Logit, power. Other transformations can be evaluated in order to have normally distributed variables.

The variables thus processed were evaluated through the calculation of the diagnostic parameters of the model R² and Q² which were very good results equal to 0.996 and 0.984, respectively. The model, therefore, was made up of 1 parallel component and 2 orthogonal components, otherwise written as [1+2], as can be observed in FIG. 1 (OPLS_NIR &Effetto sticker_Graph of the diagnostic parameters of the R² and Q² model for a parallel component and two orthogonal components)

The graph of FIG. 2a shows the experimental values of adhesion to the inclined plane on the ordinate and those predicted on the abscissa. The graph shows the regression line which is an expression of the experimental data. In an ideal line if the regression coefficient were equal to 1, the predicted value would be equal to the experimental value. Since the theoretical data always have a deviation compared to the experimental values, through the calculation of the regression coefficient R² and the calculation of the root mean square error of estimate, RMSEE (Root Mean Square Error of Estimation) it is possible to have an estimate of the goodness of the model and the deviation of the predicted value compared to the experimental value.

The model reported in the graph of FIG. 2a therefore expresses a high degree of correlation between the samples and therefore a high degree of prediction, the R² value being very high, >0.99, and the RMSEE (Root Mean Square Error of Estimation), equal to 2.0, on a scale of 70. In FIG. 2b the model is used to predict the adhesion effect to the inclined plane of the TARGET sample starting from the NIR fingerprint. As can be seen, the prediction is very good as the R^{2 value is very high,}>0.99, as is the mean square prediction error, RMSEP (Root Mean Square Error of Prediction), equal to 3.1 on a scale of 70.

In summary, FIG. 2 shows the OPLS scattered plots of the NIR fingerprint data integrated with the adhesion values to the plane where the correlation between the experimental adhesion effect (ordinate) VS the predicted adhesion effect (abscissa) is observed.) (FIG. 2a) and the prediction of the adhesion effect of the Target starting from the NIR fingerprint (FIG. 2b).

The graphs in FIG. 2 show that when projecting a new sample, for example a production batch or samples from product reformulation studies, the estimate of the effect value will be very accurate as the error is very low.

3.2. OPLS Correlation Integration of NIR Fingerprint Data with Barrier Effect Data

Fingerprint data were pre-processed using the SNV (Standard Normal Variate) algorithm and scaled using the MEAN-CENTERING function. The data relating to the barrier effect measurement were transformed using the squared exponent function and scaled using the UV (Unit Variance) function. In general, transformation algorithms can be exponential or logarithmic, such as: log, NegLog, Logit, power. Other transformations can be evaluated in order to have normally distributed variables.

The variables thus processed were evaluated through the calculation of the diagnostic parameters of the model R² and Q² which were very good results equal to 0.994 and 0.954, respectively. The model, therefore, was made up of a parallel component and four orthogonal components, otherwise written as [1+4], as can be observed in FIG. 3 (OPLS NIR&Effect barrier_Graph of the diagnostic parameters of the R² and Q^{2 model} for one parallel component and four orthogonal components).

In the graph of FIG. 4a values of the experimental barrier effect are shown on the ordinate and those predicted on the abscissa. The graph shows the regression line which is an expression of the experimental data. In an ideal line if the regression coefficient were equal to 1, the predicted value would be equal to the experimental value. Since the theoretical data always have a deviation compared to the experimental values, through the calculation of the regression coefficient R² and the calculation of the root mean square error of estimate, RMSEE (Root Mean Square Error of Estimation) it is possible to have an estimate of the goodness of the model and the deviation of the predicted value compared to the experimental value.

The model shown in the graph of FIG. 4a therefore expresses a high degree of correlation between the samples and therefore a high degree of prediction, the R² value being very high, >0.99, and the RMSEE (Root Mean Square Error of Estimation), equal to 1.0, on a scale of 100. In FIG. 4b the model is used to predict the barrier effect of the TARGET sample starting from the NIR fingerprint. As can be seen, the prediction is very good as the R² value is very high, >0.98, as is the mean square prediction error, RMSEP (Root Mean Square Error of Prediction), equal to 3.7 on a scale of 100.

In summary, FIG. 4 shows the OPLS scattered plots of the NIR fingerprint data integrated with the barrier effect values where the experimental barrier effect (ordinate) VS the predicted barrier effect (abscissa) is observed (FIG. 4a) and the prediction of the barrier effect of the Target starting from the NIR fingerprint (FIG. 4b).

The graphs in FIG. 4 show that when projecting a new sample, for example a production batch or samples from product reformulation studies, the estimate of the effect value will be very accurate as the error is very low.

3.3. Integration by OPLS Correlation of UHPLC-qToF Fingerprint Data with Antioxidant Effect Data

UHPLC-qToF fingerprint data were scaled using the PARETO function and the MEAN-CENTERING function to have a normal distribution of the variables. The data relating to the antioxidant effect measurement were not transformed but only scaled using the UV (Unit Variance) function. In general, transformation algorithms can be exponential or logarithmic, such as: log, NegLog, Logit, power. The other transformations can be evaluated in order to have normally distributed variables.

The variables thus processed were evaluated through the calculation of the diagnostic parameters of the model R² and Q² which were very good results equal to 0.959 and 0.929, respectively. The model, therefore, was made up of a parallel component and zero orthogonal components, otherwise written as [1+0], as can be observed in FIG. 5 (OPLS UHPLC-qToF & Antioxidant effect_Graph of the diagnostic parameters of the R² and Q² model for one parallel component and zero orthogonal components).

The graph of FIG. 6a shows the values of the experimental antioxidant effect on the ordinate and those predicted on the abscissa. The graph shows the regression line which is an expression of the experimental data. In an ideal line if the regression coefficient were equal to 1, the predicted value would be equal to the experimental value. Since the theoretical data always have a deviation compared to the experimental values, through the calculation of the regression coefficient R² and the calculation of the mean square error, RMSEE (Root Mean Square Error of Estimation) it is possible to have an estimate of the goodness of the model and the deviation of the predicted value compared to the experimental value.

The model reported in the graph of FIG. 6a therefore expresses a high degree of correlation between the samples and therefore a high degree of prediction, the R² value being very high, >0.95, and the RMSEE (Root Mean Square Error of Estimation), equal to 297, on a scale of 4000. In FIG. 6b the model is used to predict the antioxidant effect of the TARGET sample starting from the UHPLC-qToF fingerprint. As can be seen, the prediction is very good as the R² value is very high, >0.95 and the mean square prediction error, RMSEP (Root Mean Square Error of Prediction), equal to 347 on a scale of 4000. In summary, FIG. 6 shows the OPLS scattered plots of the UHPLC-qToF fingerprint data integrated with the adhesion values to the surface where the correlation between the experimental antioxidant effect (ordinate) VS the predicted antioxidant effect is observed (abscissa) (FIG. 6a) and the prediction of the antioxidant effect of the Target starting from the UHPLC-qToF fingerprint (FIG. 6b).

The graphs in FIG. 6 show that when projecting a new sample, for example a production batch or samples from product reformulation studies, the estimate of the effect value will be very accurate as the error is very low.

1. A process for the characterization of a product having therapeutic and/or health-promoting properties, said product comprising complex natural systems, comprising the following steps:
a. carry out a qualitative-quantitative fingerprint analysis of a standard formulation of said product and of formulations that differ from said standard formulation due to the presence of the same ingredients of the same quality at different percentages, or due to the presence of the same ingredients of different quality at the same percentages, thus obtaining a block
b. carry out an analysis of one or more biophysical properties of a standard formulation of said product and of formulations that differ from said standard formulation due to the presence of the same ingredients of the same quality at different percentages, or by the presence of the same ingredients of different quality at same percentages, thus obtaining a block Y of biophysical property data,
c. carry out an integration by correlation of the data by means of multivariate analysis which involves the correlation of said data block X with said data block Y, integrating by correlation each of the data obtained in a. with each of the data obtained in b, thus obtaining a regression line for each pair of values for which the regression coefficient R² and the mean squared estimate error RMSEE are calculated, selecting those pairs of data wherein R²≥0.8 thus providing a model that allows predict each biophysical property of the standard formulation starting from the fingerprint used.

2. A process for the production validation of one or more batches of a product having therapeutic and/or healthy properties comprising complex natural systems, including the following steps:
a. carry out a qualitative-quantitative fingerprint analysis of a standard formulation of said product and of formulations that differ from said standard formulation due to the presence of the same ingredients of the same quality at different percentages, or due to the presence of the same ingredients of different quality at the same percentages, thus obtaining an X block of fingerprint data
b. carry out an analysis of one or more biophysical properties of a standard formulation of said product and of formulations that differ from said standard formulation due to the presence of the same ingredients of the same quality at different percentages, or by the presence of the same ingredients of different quality at same percentages, thus obtaining a block Y of biophysical property data,
c. carry out an integration by correlation of the data by means of multivariate analysis which involves the correlation of said data block X with said data block Y, integrating by correlation each of the data obtained in a. with each of the data obtained in b, thus obtaining a regression line for each pair of values for which the regression coefficient R² and the mean squared estimate error RMSEE are calculated, selecting those pairs of data wherein R²≥0.8 thus providing a model that allows predict each biophysical property of the standard formulation starting from the fingerprint used;
d. carry out a qualitative-quantitative fingerprint analysis with the same methods used in a. of one or more batches of said product to be validated, using the regression model developed in c. so as to predict the biophysical characteristics described in point b.

3. The process according to any one of claim 1 or 2 wherein said qualitative-quantitative fingerprint analysis is carried out by recording the NIR spectrum, recording the IR spectrum; or by acquisition of chromatograms chromatographic systems coupled to mass spectrometers such as: gas chromatography, liquid chromatography and qToF, single quadrupole, triple quadrupole, ion trap; or by mass spectrometry by direct infusion into the aforementioned mass spectrometers, NMR, UV-Vis spectroscopy also coupled to chromatographic systems, liquid chromatography.

4. The process according to any one of claims 1 to 3 wherein said one or more biophysical properties are selected from: mucoadhesion capacity, barrier effect, antioxidant effect, neutralizing activity, hydrating activity, lubricating activity.

5. The process according to any one of claims 1 to 4 wherein said integration and correlation in c. is carried out using PLS, OPLS or PLS2.

6. The process according to any one of claims 1 to 5 wherein said product having therapeutic properties comprising complex natural systems is in the form of a medical device constituted by natural substances and wherein said product having healthy properties is in the form of a food supplement constituted by substances natural.

7. The process according to any one of claims 1 to 6 wherein said natural substances are selected from parts of plants, powders of such parts, extracts of plants or their parts such as leaves, flowers, roots, bark, stem, seeds, fruits, petioles, parts of flowers, fractions of said extracts, essential oils, natural gums, natural resins, minerals, honey, propolis, animal substances, mineral substances.

8. The process according to any one of claims 1 to 7 wherein said product is in the form of syrup, tablet, capsule, lozenge, granules, powder, solution, suspension, aerosol, powder for aerosol, powder in sachet, granules in sachet, operculum, spray, cream, emulsion, soft or hard gelatin capsule.

9. The process according to any one of claims 1 to 8 wherein said R² is ≥0.9.

10. The process according to any one of claims 1 to 9 wherein said integration by correlation of the data in c. is carried out using OPLS.
